(19) United States
(12) Patent Application Publication
(10) Pub. No.: US 2009/0180950 A1
(43) Pub. Date: Jul. 16, 2009

(54) Polychromatic, diversely-sized particles for angiography

(75) Inventors: Samir R. Tari, New York, NY (US); Gaetano R. Barile, Hawthorn, NJ (US); Uday B. Kompella, Omaha, NE (US)

Correspondence Address: SCHWEGMAN, LUNDBERG & WOESSNER, P.A., P.O. BOX 2938, MINNEAPOLIS, MN, 55402, US

(73) Assignees: The Trustees of Columbia University in the City of New York, New York, NY (US); Board or Regents of the University of Nebraska Varner Hall, Lincoln, NE (US)

(21) Appl. No.: 12/319,369

(22) Filed: Jan. 6, 2009

Related U.S. Application Data
(63) Continuation of application No. PCT/US2007/015546, filed on Jul. 6, 2007.
(60) Provisional application No. 60/806,711, filed on Jul. 6, 2006.

(51) Int. Cl. A61K 51/00; A61K 49/00; A61K 9/127; A61K 9/14; A61K 9/16

(52) U.S. Cl. 424/1.11; 424/9.1; 424/450; 424/489; 424/490; 424/499; 977/773; 977/927

(57) ABSTRACT

The invention involves polychromatic particles of various sizes for assessing blood flow, blood barrier leakage and blood vessel leakage.

### RELATED APPLICATIONS

This application is a nationalization under 35 U.S.C. 111(a) of International Application No. PCT/US2007/015546, filed Jul. 6, 2007 and published as WO 2008/005514 on Jan. 10, 2008, which claimed priority under 35 U.S.C. 119(e) to U.S. Provisional Ser. No. 60/806,711, filed Jul. 6, 2006; which applications and publication are incorporated herein by reference and made a part hereof.

### FIELD OF THE INVENTION

The invention relates to compositions useful for angiography and methods for detection of vascular leakage and blood flow. In some embodiments, the compositions and methods of the invention can be used to assess the integrity of the blood-retinal barrier and/or quantify the degree of breakdown in the blood-retinal barrier. Thus, the inventive compositions can be used as contrast materials for two dimensional, three dimensional imaging and similar techniques.

### BACKGROUND OF THE INVENTION

Retinal angiography is a technique used in ophthalmology for the study of physiological and pathological conditions of the eye. For example, angiography has been proven useful in research and clinical areas for the evaluation of retinal diseases such as diabetic retinopathy and age related macular degeneration, two of the leading causes of blindness. Currently, a fluorescent material, such as fluorescein, is administered intravenously into a patient and the retina of the patient is observed by angiographic procedures. In the healthy eye, ocular barriers prevent the leakage of fluorescein through the retinal vessels into the vitreous or other ocular tissues. However, in a diseased eye, the blood retinal barrier breaks down, resulting in the leakage of the fluorescein into the vitreous in the case of diabetic retinopathy or into the subretinal space in the case of age related macular degeneration.

One limitation of currently employed angiographic procedures is that the fluorescent material employed provides only an “all or none” assessment of the presence of leakage, or in the best case, a subjective assessment of the blood retinal barrier breakdown. Quantification of the amount of breakdown in blood-retinal and blood-brain barriers would provide better assessment of disease progression, improve our ability to choose the appropriate treatment and dosage, and allow monitoring of the effects of such treatments and dosages.

### SUMMARY OF THE INVENTION

The present invention relates to polychromatic angiography (PCA) using different labels or dyes attached, adsorbed or encapsulated onto or within differently-sized particles, beads, colloids or soluble conjugates of the label. Such particles, beads, colloids and soluble conjugates of the label/dye are collectively referred to as particles herein. A set of one size of particles is distinguishable from another set of particles of a different size by their labels or dyes. A combination of multiple sets of particles can be used to specifically detect and quantify leakage or breakdown of blood barriers. Thus, the sizes of particles that leak from blood barriers provide a measure of the degree of blood barrier breakdown. When larger as well as smaller particles leak, greater breakdown or dysfunction of the blood barrier exists, whereas leakage of only very small particles indicates that lesser breakdown or dysfunction exists (see, e.g., FIG. 4).

Thus, one aspect of the invention is a composition comprising a series of particle groups, each particle group having a different mean diameter and a distinct label that provides a distinct signal (e.g., a distinct fluorophore that absorbs or emits light at a distinct wavelength).

In general, the particles in each particle group in the composition are soluble in an aqueous environment. In some embodiments, biodegradable particles are used in the particle groups. In other embodiments, the particles are made from non-biodegradable materials, or a combination of biodegradable and non-biodegradable materials. For example, the particles can be made from poly(lactide), poly(glycolide), poly(lactide-co-glycolide) (PLGA), polyalkylene glycol, poloxamer, polyvinylpyrrolidene, methacrylate, peptides, proteins, proteinoid microspheres, lipids, liposomes or polysaccharides. Polysaccharides that can be used in the particles of the invention include cellulose and derivatized cellulose (e.g., cellulose with a variety of lower alkyl substituents), or other polymers of sugars, malate, succinate, citrate, isocitrate, α-ketoglutarate, fumarate, and the like. The particles can also contain polymers such as hydroxypropyl methacrylate, homopolymers or mixed polymers of maleic anhydride, succinate anhydride and the like.

The particles in the different particle groups have different molecular weights or sizes. For example, the particles can be as small as 500 daltons, or 800 daltons, or 1000 daltons or 5000 daltons. The particles can also have molecular weights as large as 100,000,000 daltons or 1,000,000,000 daltons. In some embodiments, the particles have diameters of up to three micrometers. In other embodiments, the particles can, for example, have diameters ranging in size from about 3 picometers to about 3 micrometers, or about 10 picometers to about 2.5 micrometers, or about 100 picometers to about 2 micrometers, or about 1 nanometer to 1 micrometer. In some instances, the particles are, at least about 10 picometers, at least about 100 picometers, at least about 1 nanometer and/or at least about 10 nanometers in size.

Different dyes and labels are used on the particles in the particle groups. Thus, for example, the particles can have labels that are fluorescent, luminescent, infrared, magnetic, radioactive or a combination thereof. Examples of fluorescent labels include fluorescein, fluorescein isothiocyanate, indocyanine green, rhodamine red, pacific blue, texas red, alexa-532, hydroxycoumarin, aminocoumarin, methoxycoumarin, amino methylcoumarin, cascade blue, lucifer yellow, P-phycoerythrin, R-phycoerythrin, lissamine rhodamine B, allophycocyanin, oregon green, tetramethylrhodamine, dansyl, monochlorobimane, fluorescent proteins, calcein or other dyes or labels attached onto, adsorbed onto or encapsulated within them. In other embodiments, the label for at least one particle group is chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III), erbium (III), lanthanum (III), gold (III), lead (II), bismuth (III), iodine¹³¹, iodine¹²³, iodine¹²⁵, technicium⁹⁹, indium¹¹¹, phosphorus³², rhenium¹⁸⁸, rhenium¹⁸⁶, gallium⁶⁷, sulfur³⁵, copper⁶⁷, yttrium⁹⁰, tritium³ or astatine²¹¹.

Another aspect of the invention is a method for quantifying blood vessel leakage in a mammal that comprises: (a) administering the particle composition of the invention to the mammal; (b) observing whether a signal is exterior to the mammal's blood vessels; and (c) if a signal is emitted exterior to the mammal's blood vessels, determining the type of signal emitted exterior to the mammal's blood vessels to quantify the blood vessel leakage in the mammal. In some embodiments, the blood vessel leakage comprises leakage through a mammal's blood retinal barrier. For example, when observing or quantifying blood vessel leakage through the blood retinal barrier, the signal can be a fluorescent signal and determining the type of signal emitted can include determining the signal's absorption or emission wavelength. In other embodiments, the blood vessel leakage can include leakage through a mammal's blood-brain barrier. When observing or quantifying blood vessel leakage through the blood-brain barrier, the signal can be a paramagnetic or radioactive signal.

The method for quantifying blood vessel leakage in a mammal can also include a step of quantifying the blood vessel leakage by correlating the signal with the size range of the particle group emitting that signal to identify a pore size in the blood vessel that gives rise to the leakage, and assigning a numerical value to that pore size.

Another aspect of the invention is a method for quantifying blood vessel leakage in a mammal that comprises: (a) administering a polychromatic particle composition of the invention to the mammal; (b) observing whether a chromatic signal is emitted exterior to the mammal's blood vessels; and (c) if a chromatic signal is emitted, determining the chromatic signal's absorption or emission wavelength to quantify the blood vessel leakage in the mammal; wherein the polychromatic particle composition comprises a series of particle groups, each particle group having a different mean diameter and a distinct chromatic signal. If a chromatic signal is emitted exterior to the mammal's blood vessels, one or more chromatic particles have extravasated (i.e., leaked) from the blood vessels. In some embodiments, for example, the blood vessel leakage comprises leakage through a mammal's blood-brain barrier. In other embodiments, for example, the blood vessel leakage comprises leakage through a mammal's blood retinal barrier. The methods of the invention can further include quantifying the blood vessel leakage by correlating chromatic signal's absorption or emission wavelength with the size range of the particle group emitting that wavelength to identify a pore size in the blood vessel that gives rise to the leakage, and assigning a numerical value to that pore size. After identifying the pore size, appropriate therapeutic agents and/or procedures can be administered and/or performed.

Another aspect of the invention is a method for observing blood flow through a blood vessel in a mammal that comprises: (a) administering a labeled particle composition of the invention to the mammal; (b) observing a signal within the mammal's blood vessels; and (c) determining the signal's type to identify a rate of blood flow and/or a size of the blood vessel in the mammal; wherein the labeled particle composition comprises a series of particle groups, each particle group having a different mean diameter and a distinct label or signal. This method can be adapted to permit identification of blood flow problems such as partial or complete blockage of a blood vessel. The extent of blockage, or the diameter of the blood vessel can be determined by observing what type of label or signal is present in the blood vessel. Blockages in blood vessels can be identified by observing that larger sized particles are prevented from flowing through the blood vessel at a distinct point.

Another aspect of the invention is a kit or article of manufacture, comprising the composition comprising a series of particle groups, each particle group having a distinct diameter size range and a distinct label or dye (e.g., a fluorophore that absorbs or emits light at a distinct wavelength), and instructions for using the composition. In some embodiments, the instructions describe how to detect and/or quantify blood vessel leakage in a mammal. In other embodiments, the instructions describe how to detect blood flow, blood vessel diameter and/or blood vessel blockage (either partial or substantially complete blood vessel blockage). The kit can also contain other useful tools such as a syringe, needle, swab, catheter, or antiseptic solution.

Another aspect of the invention is a method for observing blood flow and/or blood flow rate in a blood vessel of a mammal that includes administering one of the compositions of the invention to a blood vessel of the mammal and detecting at least one signal from at least one particle group of the composition in the blood vessel. The method can also include correlating the signal with the size range of the particle group emitting that signal to identify diameter of the blood vessel. In addition, the method can include identifying whether the diameter of the blood vessel changes along the length of the blood vessel or at a later time. Such a method can also include identifying whether the blood vessel has a partial blockage.

### DESCRIPTION OF THE FIGURES

FIG. 1A-D illustrates the properties of angiography of the retinal blood vessels performed using sodium fluorescein (102) according to currently available procedures, where the molecular weight and the effective size of fluorescein (102) is 376 daltons. Sodium fluorescein (102) has an orange-brown color, a maximum λ_{abs} of 492 nm, and a maximum λₑₘ of 518 nm. The retina (106) divides the choroid containing the outer retinal blood vessels from the inner retinal blood vessels. FIG. 1A is a diagram showing a mixture (100A) of fluorescein and plasma proteins (104), illustrating that 20-30% of fluorescein molecules (102) are unbound while 70-60% are bound to plasma proteins (104). FIG 1B illustrates the behavior of fluorescein (102) in the normal retina (100B) and shows that free fluorescein molecules (102) leak from the choroidal vessels into the choroid giving rise to background fluorescence. FIG. 1C shows leakage from the choroid or outer blood-retinal barrier (100C) while FIG. 1D shows leakage from the inner (100D) blood-retinal barrier. Thus, FIGS. 1C & D illustrate different fluorescein leakage patterns and different degrees of blood-retinal barrier dysfunction.

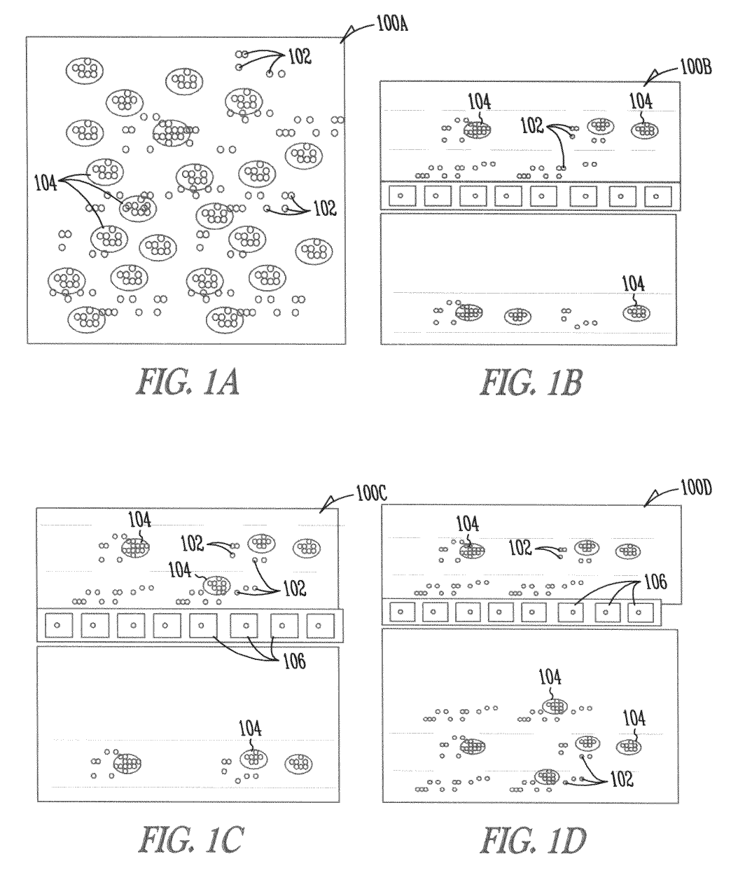

FIG. 2A-D illustrates angiography performed by indocyanine green (202; ICG) according to currently available procedures and demonstrates the concept of effective size (30-70 KDaltons), where 98% of the small indocyanine green molecules (202), which have a molecular weight of only 775 daltons, are bound to larger proteins, lipoprotein and phospholipids (204). Note that indocyanine green (202) has a green color with a maximum λ_{abs} of 800 nm, and a maximum λₑₘ of 825 nm. In this diagram, the retina (206) divides the choroid containing the outer retinal blood vessels from the inner retinal blood vessels. FIG. 2A shows a mixture (200A) of indocyanine green molecules (202) and plasma proteins and other plasma particles (204), illustrating that almost all of the indocyanine green molecules (202) are bound to plasma proteins and other plasma particles (204), and that these large proteins (204) generally prevent the leakage of the ICG (202) from the blood-retinal barrier in the normal retina, as shown in FIG. 2B. FIG. 2C shows leakage from the choroid or outer blood-retinal barrier (200C) while FIG. 2D shows leakage from the inner (200D) blood-retinal barrier. Thus, FIGS. 1C & D illustrate different fluorescein leakage patterns and different degrees of blood-retinal barrier dysfunction.

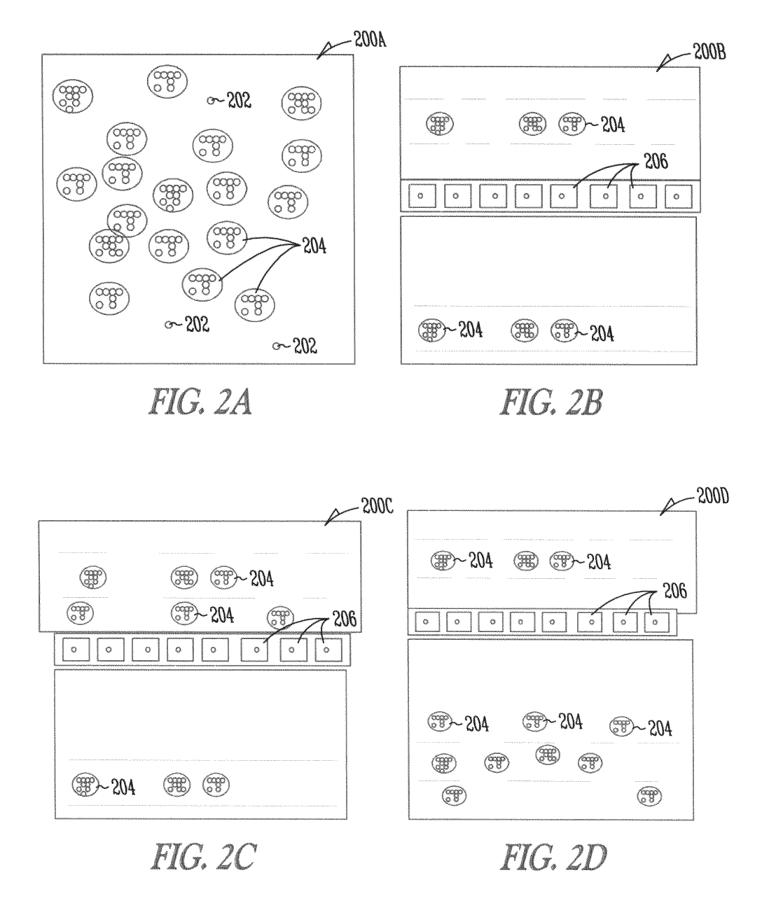

FIG. 3 illustrates one embodiment of the invention—a composition (300B) containing a series of fluorophores bound to different sized particles (302, 312, 314 and 316), where the particles are represented by larger white circles and the fluorophores are represented by small dots (302, 304, 306 and 308). As illustrated, the final composition is made from a series of separate mixtures (300A) of different fluorophores (302, 304, 306 and 308) with different sized particles. After mixing and binding the fluorophores to the particles (310), three fluorophores are bound to a different sized particle (312, 314, 316, having sizes ranging, e.g., from about 500 daltons to 1 micron). One fluorophore (302) is unbound and constitutes the smallest particle species. Thus, the composition (300B) comprises a multitude of particles species, each of controlled size, each with a different label (or fluorophore)(302, 312, 314, 316), where the label or fluorophore type signals the size of the bead.

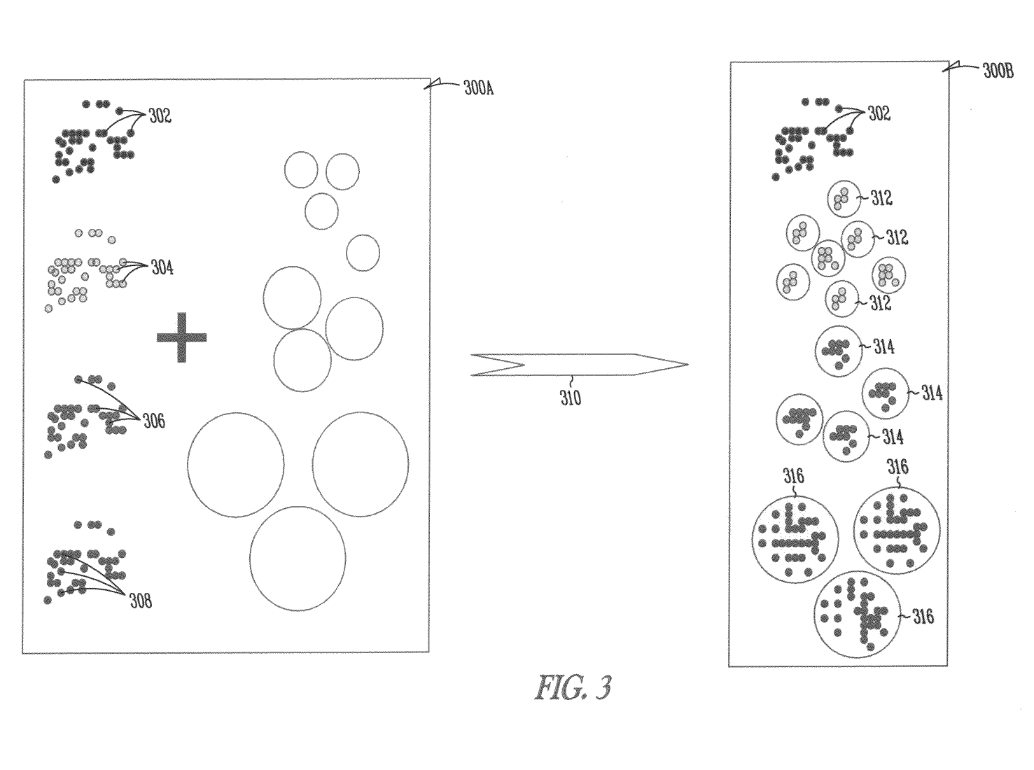

FIG. 4 illustrates the utility of the present compositions for assessing the degree of dysfunction in blood vessels or the degree of dysfunction in various blood barriers such as blood-retinal barriers. The composition (300B) employed has the same labeled particles (302, 312, 314, 316) illustrated in this schematic diagram are the same as those shown in FIG. 3. Thus, in a normal healthy blood vessel (400A), substantially no labeled particles escape the blood vessel, essentially no dysfunction exists and the degree of dysfunction can, for example, be labeled stage 0 or (No dysfunction). If minimal dysfunction exists (400B), very small beads (302) escape a blood vessel. This indicates that some small degree of dysfunction exists that can, for example, be graded as minimal or stage 1 dysfunction. If moderate dysfunction exists (400C), slightly larger beads as well as the very small particles (302 and 312) escape a blood vessel. This indicates that a somewhat greater degree of dysfunction exists that can, for example, be labeled moderate or stage 2 dysfunction. If severe dysfunction exists (400D), somewhat larger particles (314) as well as the smaller previously mentioned particles (302 and 312) escape a blood vessel. This indicates that a greater degree of dysfunction exists that can, for example, be labeled severe or stage 3 dysfunction. If very severe dysfunction exists (400E) quite large particles (316) as well as the smaller particles escape a blood vessel (302, 312 and 314), an even greater degree of dysfunction exists that can, for example, be labeled very severe or stage 4 dysfunction.

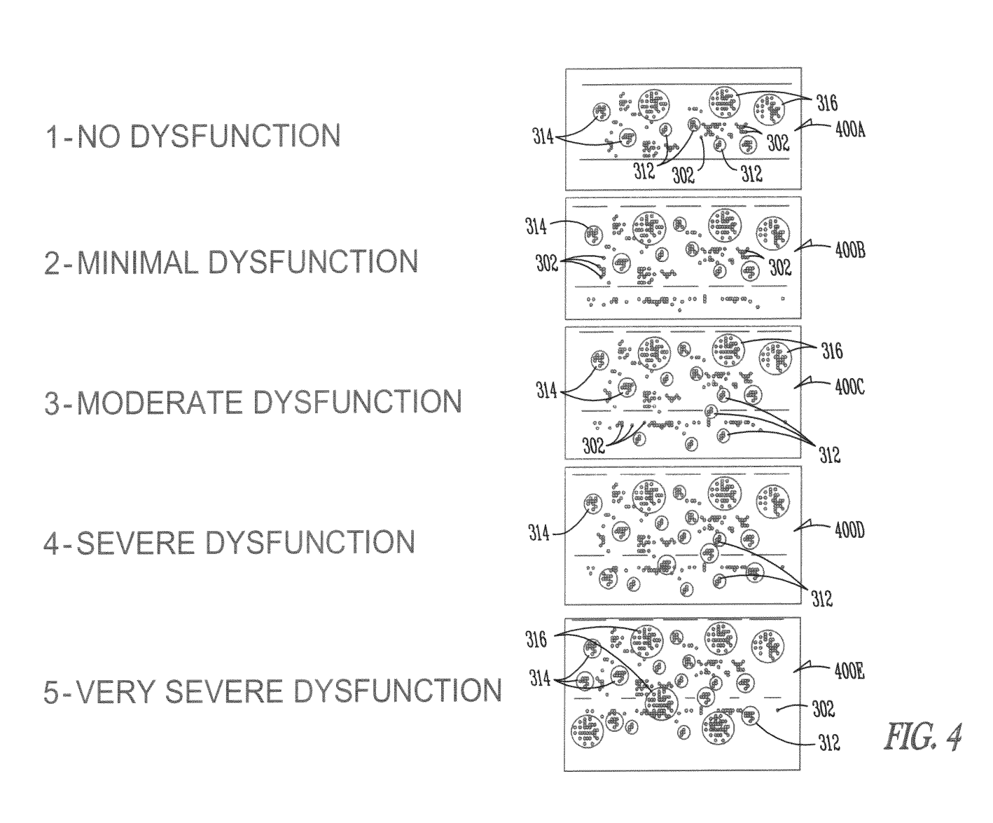

FIG. 5 illustrates an in-vitro experiment that shows separation of different sized particles preparations with different chromophores, where separation was achieved by filtration through membranes with 0.2 micron pore sizes. Three sets of dyes/mixtures were made consisting of fluorescein bound to PLGA particles that were less than 0.2 microns in size, indocyanine green (ICG) bound to PLGA particles that were greater than 0.2 microns in size, and a mixture of both types of particles. Two aliquots of each set were tested. One aliquot was filtered through a 0.2 micron filter and the second aliquot was not filtered. The bottom row of images from left to right shows the non-filtered fluorescein-PLGA particles, the non-filtered fluorescein-PLGA and indocyanine green-PLGA mixture of particles and the non-filtered indocyanine green-PLGA particles. The top row of images from left to right shows the filtered fluorescein-PLGA particles (freely passing through the filter), the filtered mixture where the fluorescein-PLGA particles freely passed through the filter while the indocyanine green-PLGA particles were trapped, and the filtered indocyanine green-PLGA particles that were totally trapped by the filter. This in vitro experiment demonstrates the concept of selective leakage.

FIGS. 6A and 6B compares angiographic images obtained using currently available indocyanine green (ICG) procedures (FIG. 6A) and the present procedures with indocyanine (ICG) bound to PLGA particles (FIG. 6B). For the FIG. 6A experiment, 2.5 mg ICG in 1 ml saline was administered to rabbits and for the FIG. 6B experiment, 2.5 mg ICG particles (3.3% ICG load) in 1 ml saline was administered to rabbits. The in vivo behavior of the dye and/or particles in the rabbit retina was then observed. As shown in FIG. 6B, the particle-bound ICG gave rise to brighter, more distinct images. Because free ICG binds to tissues whereas the particle-bound ICG does not, use of particle-bound ICG provides improved, clear, distinct images of blood vessels.

FIGS. 7A and 7B compares angiographic images obtained using currently available free fluorescein angiography (FIG. 7A) or the present procedures that employ fluorescein bound to particles (FIG. 7B). The two fluorescein preparations were administered to rabbits and the in vivo behavior of the dye/particles in the rabbit retina was observed. As shown, the particle-bound fluorescein gave rise to images without significant background. Because free fluorescein binds to tissues whereas the particle-bound fluorescein does not, use of particle-bound fluorescein provides improved, clear, distinct images of blood vessels without background fluorescence.

FIG. 8A-F illustrates the selective leakage of different-sized labeled groups in the retina after administration of mixtures of compositions to rabbits. FIG. 8A-C are angiographic images of healthy retinal blood vessels after administration of a mixture of ICG-bound particles and free fluorescein. In FIG. 8B the ICG-bound particles were detected while in FIG. 8C, the fluorescein-bound particles were detected. FIG. 8A shows the fluorescence detected from both ICG-bound particles and free fluorescein in the normal rabbit retina. FIG. 8D-F are angiographic images of mildly dysfunctional retinal blood barriers after administration of a mixture of ICG-bound particles and free fluorescein to a rabbit. Mild retinal blood barrier dysfunction was induced in rabbits by induction of Uvietis through intravitreous injection of LPS (lipopolysaccharide 20 nanograms). In FIG. 8E the ICG-bound particles were detected while in FIG. 8F, the free fluorescein was detected. FIG. 8D shows the fluorescence detected from both ICG-bound particles and free fluorescein in the mildly dysfunctional rabbit retinal blood barrier. Note that in the normal rabbit retina (FIGS. 8A-C) none of the dyes leaked, while in the mildly dysfunctional blood-retinal barrier model, the smaller unbound fluorescein particles leaked from the retinal vessels (the green color in FIGS. 8D and 8F) while the particle-bound ICG (red in FIGS. 8D & E) did not leak.

### DETAILED DESCRIPTION OF THE INVENTION

The invention involves a composition of different groups of distinctly sized and distinctly labeled particles or beads. The present compositions can be used in methods to quantitatively assess the integrity of blood barriers (e.g., the blood retinal barrier) by detecting the size of particles found outside the blood barrier (e.g., in the vitreous). The size of the particle is identified by observing which type of dye or label is present on the exterior of the blood barrier. The present compositions can also be used in methods to assess or detect blood flow through blood vessels (e.g., and identify partial or complete blockages in blood flow, or diminished rates of blood flow due to systemic or localized problems) by detecting the rate and size of particles flowing through blood vessels. Thus, partial blockage of blood vessels can be detected by observing whether the size of particles passing through a blood vessel changes abruptly. To detect these occurrences (e.g., leakage, partial blockage) different dyes or labels are attached onto, adsorbed onto or loaded into particles of different sizes to generate the compositions of the invention.

According to the invention, the degree of disruption in a blood barrier is quantitatively assessed by observing the degree to which larger particles escape the blood barrier. For example, only smaller particles leak in case of mild dysfunction of the blood retinal barrier (BRB) while the larger sized particles leak when BRB dysfunction is more severe. The size of the particles is detected by observing which labels or dyes escape the blood barrier. The severity of blood barrier leakage or dysfunction can be graded by size of particle leaked from the blood barrier. The leaking particles therefore reflect the pore size(s) in the blood barrier. The presence of most of the composition particles in the blood vessels is evidence that the blood vessels are substantially intact. Accordingly, therapeutic agents and procedures can be used to appropriately treat the detected degree of blood vessel leakage.

The blood flow through blood vessel (and possible blockages in blood flow) can also be assessed using the particle compositions of the invention. Thus, when observing blood flow using the present compositions, a mixture of larger particles and smaller particles will be detected in larger blood vessels but smaller particles may be observed in smaller blood vessels. However, if a blockage has occurred in the larger blood vessels, a change in the size of particles will be observed downstream from the blockage. In particular, a greater proportion of smaller particles will be observed downstream of the blockage than was observed upstream of the blockage.

### Particles

As indicated above, the compositions of the invention contain labeled particles of diverse sizes. In particular, the compositions contain a series of particle groups, each particle group having a mean diameter that is different from the mean diameters of other particle groups in the composition. Moreover, each particle group has a distinct label that emits a distinctive signal that is readily distinguishable from the labels/signals of other particle groups in the composition.

The particle group sizes are selected to permit assessment of the severity of blood barrier or vascular leakage. Thus, a variety of sizes is used. In general, about two to about twenty or about two to about ten different particle group sizes are employed in the composition. In some embodiments, about two to about eight, or about or about two to about six different particle group sizes are employed in the composition. The particle sizes can vary by molecular weight or by size (e.g. diameter). For example, the particles can be as small as 500 daltons, or 800 daltons, or 1000 daltons or 5000 daltons. The particles can also have molecular weights as large as 100,000,000 daltons or 1,000,000,000 daltons. In some embodiments, the particles have diameters of up to about three micrometers, or up to about 2 micrometers, or up to about 1 micrometer. In other embodiments, the particles can, for example, have diameters as small as about 10 picometers, or about 100 picometers, or about 1 nanometer. Thus, for example, the beads can range in size from about 10 picometers to about 3 micrometers, or about 100 picometers to about 2 micrometers or about 1 nanometer to about 1 micrometer. In some embodiments, the particles can range from about 10 picometers to 900 nanometers in diameter or about I nanometer to about 1 micrometer.

For example, in the compositions of the invention one group of particles can have a mean diameter of about 1-2 micrometer, another group of particles in the composition can have a mean diameter of about 400-600 nanometers, yet another group of particles can have a mean diameter of about 100-200 nanometers, still another group of particles can have a mean diameter of about 40-60 nanometers, a further group can have a mean diameter of about 5-20 nanometers and a final group can consist of a free label.

The particles can be made from biodegradable materials that will be gradually dissolved in the body of a living subject. Alternatively, the particles can be made from non-biodegradable materials or a combination of biodegradable and non-biodegradable materials. Moreover, materials used in the particles of the invention will not have any substantial toxic or other harmful effect on the subject. In general, the particles and/or the materials used in the particles are not sufficiently hydrophobic for absorption or adsorption onto tissues and biological molecules. Particles of the invention are preferably easily suspended or dissolved in aqueous solutions without substantial loss of label/dye or their structural integrity for the time needed to perform the diagnostic methods of the invention (e.g., about 6 to about 24 hours).

Suitable biodegradable materials can break down in vivo (e.g. by enzymatic action) or dissolve in the aqueous environment of the body. Examples of suitable biodegradable materials include polysaccharides, polyalkylene glycols, proteins, peptides, proteinoid microspheres and the like. In some embodiments, poly (D,L-lactide-co-glycolide) (PLGA), polylactic acid, polyglycolic acid, dilactic acid, and lactic acid-glycolic acid copolymers are used, which can be purchased from Poly Sciences Incorporated, Moorington, Pa. or from Birmingham Polymers (Durect Corporation, Pelham, Ala.). Other biodegradable polymers can be used as well, for example, polyalkylene glycol (e.g., polyethylene glycol), protein, proteinoid microspheres (thermally treated mixtures of amino acids), liposomes and the like.

As illustrated herein, nanoparticles of poly (D,L-lactide-co-glycolide) (PLGA) loaded with sodium fluorescein and indocyanine green have successfully been generated and used for detecting blood-retinal barrier leakage.

The concentrations of different particle groups in the present compositions can be varied as desired by one of skill in the art. In some embodiments, the compositions contain approximately the same amount of each particle group, as assessed either by particle number or label signal. Thus, the compositions can have particle groups where each particle group has approximately the same number of particles. Alternatively, when the various particle groups contain different labels with different signal strengths, the composition of particle groups can be adjusted so that each particle group emits approximately the same amount of signal, irrespective of particle number.

### Labels

A variety of labels can be used on the particles of the invention. Essentially any type of label can be used. However, it may be convenient to select labels for use on the various particle groups that can all be detected with a single device. Thus, for example, different fluorophores or luminescent molecules can be incorporated into the various particle groups, or different radioisotopes, or different metals, or different magnetic or paramagnetic atoms, or different infrared or ultraviolet absorbing or emitting molecules or different enzymes can be used with the particles.

In some embodiments, different fluorophores are used on the different particle groups. For example, the particle groups can have fluorescein, fluorescein isothiocyanate, indocyanine green, rhodamine red, pacific blue, texas red, alexa-532,hydroxycoumarin, aminocoumarin, methoxycoumarin, amino methylcoumarin, cascade blue, lucifer yellow, P-phycoerythrin, R-phycoerythrin, lissamine rhodamine B, allophycocyanin, oregon green, tetramethylrhodamine, dansyl, monochlorobimane, calcein and other fluorophore labels.

Currently, retinal angiography typically involves fluorescein angiography (FA) and/or indocyanine green angiography (ICGA). Sodium fluorescein, the dye used in FA is a 376.27 D orange-brown dye, it is 70-80% bound to plasma protein while the remainder 20-30% is free. The maximal absorption wavelength of fluorescein is 492 nm and its maximal emission wavelength is 518. On the other hand, about 98% of indocyanine green (ICG)—775 D—is bound to plasma protein, lipoprotein and phospholipids with minimal amount of unbound dye. The maximal absorption and emission wavelengths of indocyanine green are 800 nm and 825 nm, respectively. The difference in absorption and emission spectra of these two dyes play a major role in characterizing their visibility in angiography. The retinal pigment epithelial (RPE) layer blocks the short wave length luminescence of fluorescein, hence the choroidal vessels are not visible and only the retinal vessels can be seen. On the other hand, indocyanine green has a longer luminescence spectra and is not blocked by the RPE layer. These qualities permit visualization of the choroidal vessels with indocyanine green in addition to the retinal blood vessels.

The second difference between the two dyes is their binding characteristics. The kinetics of dye distribution in the retina is partially dependent on its effective size, in particular, its molecular size if unbound or the particle-dye complex size in the case of bound dyes. ICG is almost completely bound to proteins, lipoproteins and phospholipids and hence follows the distribution of these particles. On the other hand fluorescein is only 70-80% bound to plasma proteins and the remaining 20-30% of the molecules are unbound, and have the ability to leak through small pores that the bound ICG cannot escape (e.g., the leakage of fluorescein from porous choriocapillaries that are not permeable to the ICG). The binding of dyes is related to their physicochemical structure. However, when loaded onto particles the dyes loose their ability to bind to plasma proteins. Thus, when bound to the particles of the invention, the problems associated with tissue absorption of dye, including high background signals and the need to use higher doses of dye, are solved.

While fluorescent labels are useful for detection of blood vessels positioned so that the fluorophores can absorb and emit light, many blood vessels are not so conveniently located that such a fluorescent signal can be detected without invasive procedures. Thus, non-fluorescent labels and dyes can also be used on the particles of the invention that the signals of these non-fluorescent labels and dyes can be detected by non-invasive procedures such as radiography, ultrasound, magnetic resonance and the like.

For example, the particles can be labeled with radioactive labels, magnetic labels, paramagnetic labels, contrast agents, and/or gases can be incorporated into the lumen of particles.

In the case of paramagnetic ions, one of skill may choose to use, for example, ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being preferred. Ions useful in other contexts, such as X-ray imaging, include but are not limited to lanthanum (III), gold (III), lead (II), and especially bismuth (III).

In the case of radioactive isotopes for diagnostic application, one may use iodine¹³¹, iodine¹²³, iodine¹²⁵, technicium⁹⁹, indium¹¹¹, phosphorus³², rhenium¹⁸⁸, rhenium¹⁸⁶, gallium⁶⁷, sulfur³⁵, copper⁶⁷, yttrium⁹⁰, tritium³or astatine²¹¹.

The labels can be adsorbed or covalently attached to the particles by available procedures. In some embodiments, the particles are simultaneously formed and labeled. In other embodiments, the particles are manufactured and the labels are added later. Labels and dyes can be directly adsorbed or attached to the particles, or the labels and dyes can be indirectly attached via a linker or suitable attachment group.

Thus, for example, nanoparticles can be prepared using a nanoprecipitation technique (Chorny et al., 2002, Journal of Controlled Release 83:389-400 and 401-414) or modifications thereof. For example, polymer (e.g. poly (D,L-lactide-co-glycolide) (PLGA)) and label can be dissolved in a mixture of solvent (e.g. acetone, ethanol and dichloromethane), then poured into an aqueous phase of 1% polyvinylacetate (PVA) in filtered, distilled water using moderate stirring. After overnight evaporation of the organic phase, the suspension is filtered and centrifuged. The particle pellet can be resuspended in water and lyophilized. The particles can be re-suspended in a suitable physiological solvent such as water or phosphate buffered saline.

Alternatively, suitable linking groups can be added to the particles during manufacture. For example, functional groups can be used for attachment of linkers and/or labels and dyes. Functional groups that can form covalent bonds include, for example, —COOH and —OH; —COOH and —NH₂; and —COOH and —SH. For example, the linking group can conveniently be linked to the detectable label or dye through an: 1) amide (—N(H)C(═O)—, —C(═O)N(H)—), 2) ester (—OC(═O)—, —C(═O)O—), 3) ether (—O—), 4) thioether (—S—), 5) sulfinyl (—S(O)—), or 6) sulfonyl (—S(O)₂) linkage. Such a linkage can be formed from suitably functionalized starting materials using synthetic procedures that are known in the art.

### Detection of Blood Barrier Dysfunction

According to the invention, blood barrier dysfunction in a mammal can be detected and quantified by observing the escape of particles from blood vessels of the mammal. The different labels on the particles permit detection of particle escape and the different sizes of the particles that escape permits diagnosis of the extent of blood barrier dysfunction. Thus, the type and, in some embodiments, the concentration of label detected outside the blood barrier facilitates such diagnosis.

To quantify such dysfunction, numerical values can be assigned based on the type of particle that escapes. Thus, if essentially no particle escape is detected, the blood barrier can be identified as having a grade 0 or no dysfunction blood barrier. If very small particles escape from blood vessels, the blood barrier can be identified as having a grade 1 or mildly dysfunctional blood barrier. If slightly larger particles escape from blood vessels, the blood barrier can be identified as having a grade 2 or moderately dysfunctional blood barrier. If somewhat larger particles escape from blood vessels, the blood barrier can be identified as having a grade 3 or somewhat severely dysfunctional blood barrier. If even larger particles escape from blood vessels, the blood barrier can be identified as having a grade 4 or very severely dysfunctional blood barrier, and so forth.

Particles that escape from blood barriers can be detected by any convenient means. For example, particle escape can be detected by using tomography, modified tomography, modified Optical Coherence Tomography (OCT), modified con-focal scanning laser ophthalmoscopy (SLO), a modified combination device (SLOIOCT) or any other device capable of detecting the labels or dyes (e.g. fluorophores) in tissues.

Blood barrier dysfunction can be detected in a variety of tissues, for example, in the eye, the brain and other tissues. In some embodiments, the compositions and methods of the invention are used to detect blood-retinal barrier integrity and/or dysfunction.

Many types of blood barrier dysfunction can be detected by the compositions and methods of the invention. Examples of blood barrier dysfunction that can be detected include, for example, diabetic retinopathy, macular degeneration, CMV eye infection, retinitis, choroidal ischemia, acute sectorial choroidal ischemia, ischemic optic neuropathy, and other diseases.

### Methods

As indicated above, one aspect of the invention is a method for quantifying blood vessel leakage in a mammal that comprises: (a) administering a polychromatic particle composition to the mammal; (b) observing whether a chromatic signal is emitted exterior to the mammal's blood vessels; and (c) if a chromatic signal is emitted, determining the what is the chromatic signal to thereby quantify the blood vessel leakage in the mammal; wherein the polychromatic particle composition comprises a series of particle groups, each particle group having a distinct diameter size range and a distinct chromophore that provides a distinct signal. If the chromatic signal is emitted exterior to the mammal's blood vessels, one or more chromatic particles have extravasated (i.e., leaked) from the blood vessels.

The invention further provides methods for identifying agents that can modulate blood barrier integrity. Such a method can be used not only to identify beneficial agents that improve blood barrier integrity but also to assess whether an agent is toxic or leads to blood barrier dysfunction.

Thus, one aspect of the invention is a method for identifying agent that can modulate blood barrier integrity in a mammal that involves administering a test agent and a particle composition of the invention to the mammal and quantifying particle escape from blood barriers of the mammal relative to particle escape observed when only the present particle composition (without the test agent) is administered to a mammal. Test agents that increase particle escape may be toxic.

Alternatively, test agents that decrease particle escape can be beneficial therapeutic agents useful for treatment of blood barrier dysfunctional and inappropriate blood vessel leakage. Thus, in some embodiments, the mammal can have a dysfunctional blood barrier (e.g. a dysfunctional retinal blood barrier) and the method is used to screen for test agents that improve blood barrier function (i.e., inhibit particle escape). Thus, another aspect of the invention is a method for identifying agent that can promote blood barrier integrity in a mammal that involves administering a test agent and a particle composition of the invention to the mammal and quantifying particle escape from blood barriers of the mammal relative to particle escape observed when only the present particle composition (without the test agent) is administered to a mammal, wherein the mammal has a dysfunctional blood barrier. In the screening methods of the invention, mammals with dysfunctional blood barriers exhibit particle escape from blood barriers.

Another aspect of the invention is a method of detecting and/or monitoring blood flow through a blood vessel in a mammal. This method involves administering a particle composition of the invention to a mammal and observing the rate and type of particles flowing through a blood vessel in the mammal.

While the particles can be labeled with fluorescent dyes when light can be absorbed and emitted though the blood vessel(s) (e.g., retinal blood vessels), non-fluorescent labels may be used when blood vessels are not so easily observed. Thus, light may not be absorbed and emitted in a readily detectable manner from blood vessels in the brain, heart, appendages and other tissues without invasive procedures. To avoid such invasive procedures, the particles can be labeled with radioactive labels, magnetic labels, paramagnetic labels, contrast agents, and/or gases can be incorporated into the lumen of particles.

In the case of paramagnetic ions, one of skill may choose to use, for example, ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being preferred. Ions useful in other contexts, such as X-ray imaging, include but are not limited to lanthanum (III), gold (III), lead (II), and especially bismuth (III). Moreover, in the case of radioactive isotopes for diagnostic application, one may use iodine¹³¹, iodine¹²³, iodine¹²⁵, technicium⁹⁹, indium¹¹¹, phosphorus³², rhenium ¹⁸⁸, rhenium¹⁸⁶, gallium⁶⁷, sulfur³⁵, copper⁶⁷, yttrium⁹⁰, tritium³ or astatine²¹¹.

Accordingly, the compositions of the invention can be used to assess or monitor blood flow in blood vessels. For example, blood flow in the heart, brain, internal organs (e.g., liver, kidneys, intestines, stomach), and appendages can be monitored. In addition, blood flow can also be monitored in blood vessels leading to the heart, brain, internal organs (e.g., liver, kidneys, intestines, stomach), and appendages using the compositions and methods of the invention.

Mammals that can be tested, examined or diagnosed include humans and domestic animals such as rabbits, mice, rats, dogs, cats, sheep, goats, cattle, horses and zoo animals.

### Compositions

The labeled particle compositions of the invention are administered to permit analysis of the integrity and/or dysfunction of blood barriers and/or to monitor blood flow.

To permit analysis the compositions are typically administered in a single dosage or in two dosages or a divided dosage. The dosage can vary and can, for example, be at least about 0.01 mg/kg to about 500 to 750 mg/kg, of at least about 0.01 mg/kg to about 300 to 500 mg/kg, at least about 0.1 mg/kg to about 100 to 300 mg/kg or at least about 1 mg/kg to about 50 to 100 mg/kg particle composition per body weight, although other dosages may provide beneficial results. The amount administered will vary depending on various factors including, but not limited to, what types of labels used on the particles, the route of administration, the progression or lack of progression of blood barrier breakdown, the weight, the blood pressure, the physical condition, the health, and the age of the patient. Such factors can be readily determined by the clinician employing animal models or other test systems that are available in the art.

The compositions are prepared as described herein or by procedures available in the art, and purified as necessary or desired. The particle compositions can then be lyophilized or stabilized; their concentrations can be adjusted to an appropriate amount, and other agents can optionally be added. The absolute weight of a given particle group or combination thereof that is included in a unit dose can vary widely. For example, about 0.01 to about 2 g, or about 0.1 to about 500 mg, of at least two particle groups can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g of at least two particle groups.

A pharmaceutically acceptable carrier can be included in the particle groups of the invention. By “pharmaceutically acceptable” it is meant a carrier, diluent, excipient, and/or salt that is compatible with the other ingredients of the formulation, and not deleterious to the recipient thereof. Non-limiting examples of the carriers and/or diluents that are useful in the compositions of the present invention include water, aqueous sugar solutions, physiologically acceptable buffered saline solutions such as phosphate buffered saline solutions pH 7.0-8.0, and the like.

The compositions of the invention can be formulated for intravenous, intra-arterial, or intra-vascular administration.

### Kits

The present invention further pertains to a packaged composition such as a kit or other container for detecting blood barrier integrity and/or breakdown, or for detecting blood flow. The kit or container holds a composition comprising a series of particle groups, each particle group having a different mean diameter and a distinct label. Instructions are provided in the kit or container for detecting blood barrier integrity and/or breakdown of a blood barrier.

Alternatively, the kit can designed for detecting and/or monitoring blood flow and for detecting blood flow problems. The kit or container holds a composition comprising a series of particle groups, each particle group having a different mean diameter and a distinct label. Instructions are provided in the kit or container for detecting and/or monitoring blood flow and for detecting blood flow problems.

The kits of the invention can also comprise tools useful for administering the compositions of the invention. Such tools include syringes, swabs, catheters, antiseptic solutions and the like.

The following Examples further illustrate the invention and are not intended to be limiting thereof.

### EXAMPLE 1

### Materials and Methods

Materials: Poly (D,L-lactide-co-glycolide) (PLGA), M_{w}=10,000 Da (intrinsic viscosity: 0.17 dL/g) was purchased from Birmingham Polymers (Durect Corporation, Pelham, Ala.). Indocyanine green, sodium fluorescein and poly(vinyl-alcohol) (PVA) were obtained from Sigma-Aldrich (St Louis, Mo.). Dichloromethane was obtained from Acros (Morris Plains, N.J.). All other chemicals were of analytical grade and obtained from local sources.

Nanoparticle Formulation. PLGA nanoparticles were prepared using a nanoprecipitation technique (Chorny et al., 2002, Journal of Controlled Release) with some modifications. Briefly, the polymer (70 mg) and either indocyanine green or fluorescein (30 mg) were dissolved in a mixture of 15.5 ml acetone, 4 ml ethanol and 0.5 ml dichloromethane and poured into an aqueous phase of 1 % PVA in filtered, distilled water (40 ml) at moderate stirring. After overnight evaporation of the organic phase using a Rotovap (Buchi Rotavapor R200, Buchi Analytical Inc., New Castle, Del.) at 37° C. in a heated water bath (Buchi Heat Bath B490), the suspension was filtered through a 0.2 μm filter (Fisher Scientific, Pittsburgh, Pa.) followed by centrifugation at 35,000 g for 30 min at 4° C. The particle pellet was resuspended in double distilled water (20 ml) and lyophilized.

### Nanoparticle Characterization:

Particle Size Analysis. A dilute particle suspension in double distilled water was vortexed at high speed (Vortex Genie, Scientific Industries, Bohemia, N.Y.) to facilitate particle resuspension. The particle size, size distribution, and zeta potential were determined using Zeta-sizer, a particle size analyzer based on dynamic light scattering (Brookhaven Instruments Co., Holtsville, N.Y.).

Drug Loading: To 1 mg of lyophilized indocyanine green- or fluorescein-loaded PLGA particles, in a glass Kimble tube, 1 ml of methylene chloride was added and the tube was sealed. The tubes were vortexed at high speed (Vortex Genie, Scientific Industries, Bohemia, N.Y.). After 1 hour of vortexing the methylene chloride extract was evaporated using an N-Evap. The residual material was reconstituted in 1 ml of double distilled water by vortexing at high speed for 5 min. The solution was then analyzed using the UV spectrophotometer or spectrofluorometer. Controls with equivalent amounts of dye (indocyanine green or fluorescein) with or without polymer in methylene chloride were also subjected to the same treatment and the near complete recovery of the solutes was ensured.

### Animal Studies:

Male Dutch Belted rabbits were used for animal studies. The rabbits were anesthetized using a combination of Ketamine and Xylazine 35 and 5 mg/Kg body weight respectively. The right eye of each of the rabbits was dilated using Tropicamide and phenylephrine. Then fluorescein, indocyanine green, fluorescein-PLGA, indocyanine green-PLGA or combinations thereof were injected through the auricular vein. Images and videos were captured and recorded using an HRAII scanning laser ophthalmoscopy (Heidelberg Engineering, Heidelberg Germany) using the simultaneous fluorescein and indocyanine module. In some instances, retinal angiographs were further enhanced using Adobe Photoshop (7.0). All animals were treated in accordance to the guidelines of Columbia University Institutional Animal Care and Use Committee, and Association of Research in Vision and Ophthalmology.

### EXAMPLE 2

### Detecting Blood-Retinal Barrier Dysfunction

This Example shows that compositions of different-sized, differently-labeled particles can be used to detect blood-retinal barrier breakdown.

### Filtration Studies Illustrate the Different Sizes of Particles

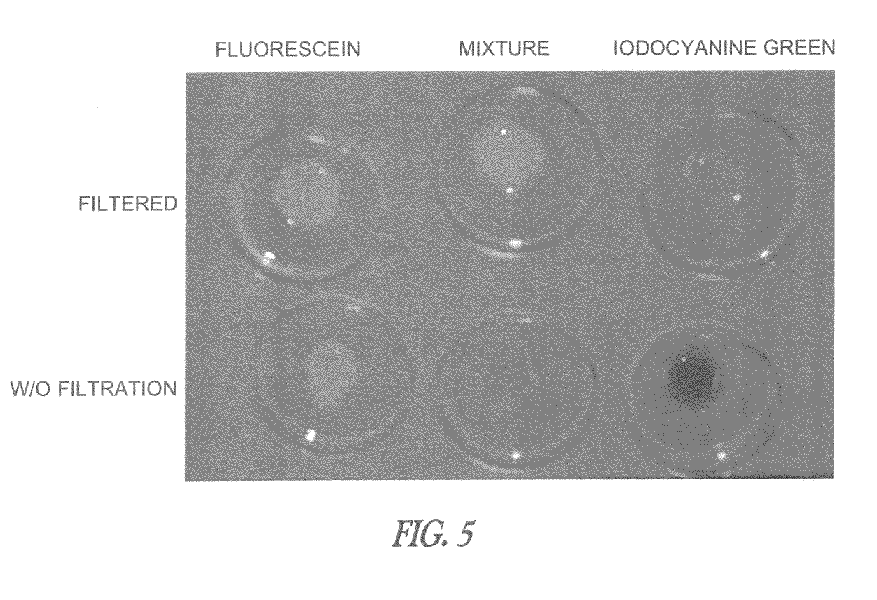

As shown in FIG. 5, different sized particle preparations with different chromophores were generated. Separation and detection of differently sized particles was effected by filtration through membranes with different pore sizes. Thus, a composition of two particle groups was prepared, as shown in FIG. 5 (bottom row, far right). This composition is shown in FIG. 5 (bottom row, center). The composition contained smaller, fluorescein-labeled PLGA particles that are shown in separated form in FIG. 5 (bottom row, left) and larger indocyanine green-PLGA particles that are shown in FIG. 5 (bottom, right). Thus, the composition contained a fluorescein-PLGA and indocyanine green-PLGA mixture and indocyanine green-PLGA.

The top row of FIG. 5 illustrates that the fluorescein-labeled particles are the smaller particles and the indocyanine green-labeled particles are the larger particles. Thus, the fluorescein-PLGA particles were freely filtered through a 0.2 micron filter (FIG. 5, top, left), while the indocyanine green-PLGA did not pass through the 0.2 micron filter (FIG. 5, top, right). When the mixture of the two particles was filtered the indocyanine green-PLGA did not pass through but the fluorescein-PLGA did (FIG. 5, top, center). Thus, the indocyanine green-PLGA particles had a mean diameter that is larger than 0.2 microns while the fluorescein-PLGA particles had a mean diameter that was smaller than 0.2 microns.

### The Present Compositions Detect Blood-Retinal Barrier Dysfunction

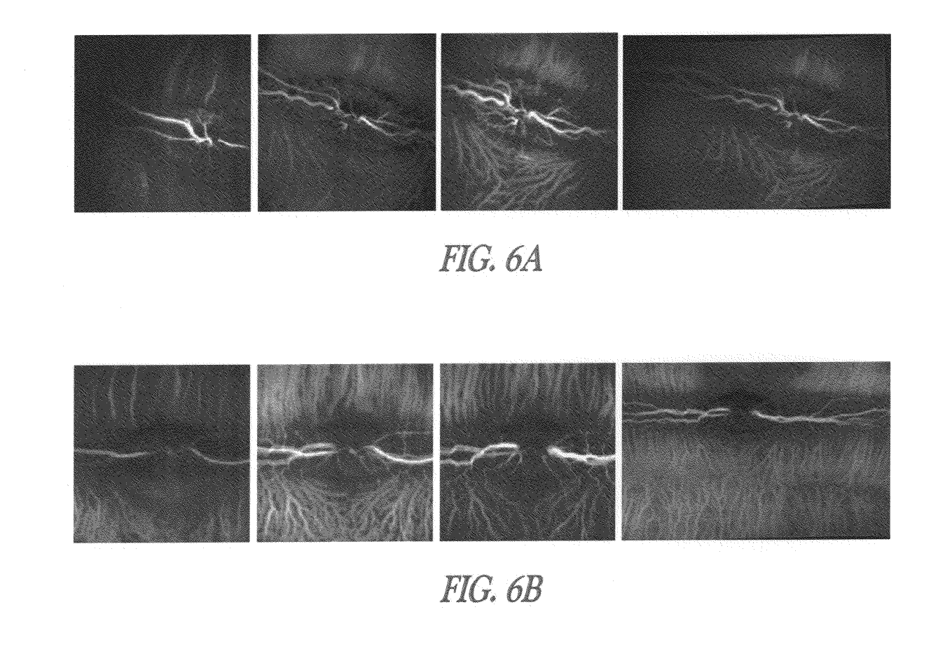

A comparison of healthy, stage 0, blood retinal barrier images as detected by the present compositions and methods versus currently available procedures is shown in FIG. 6. FIG. 6A shows a series of angiographic images obtained using currently available procedures and free indocyanine green (ICG). FIG. 6B shows a series of angiographic images obtained using the present procedures with indocyanine (ICG) bound to PLGA particles. The two ICG preparations were administered to the auricular vein of rabbits and the in vivo behavior of the ICG preparations was observed in the rabbit retina. As shown, the particle-bound ICG gave rise to brighter, more distinct images. Because free ICG binds to tissues whereas the particle-bound ICG does not, use of particle-bound ICG provides improved images of blood vessels that are clear and distinct.

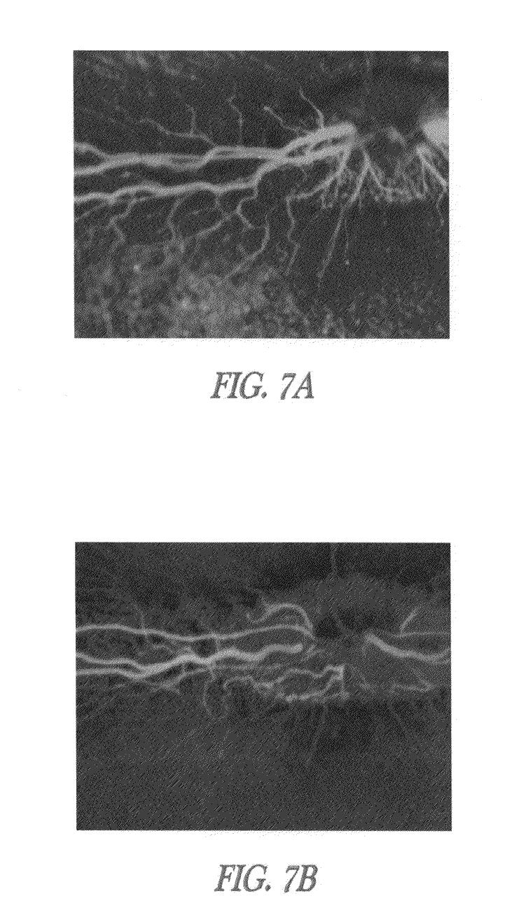

FIG. 7 shows a comparison of angiographic images obtained using currently available free fluorescein angiography (FIG. 7A) or a composition of the invention where fluorescein is bound to particles (FIG. 7B). The two fluorescein preparations were administered to the auricular vein of rabbits and the in vivo behavior in the rabbit retina was observed. The particle-bound fluorescein gave rise to images without significant background (FIG. 7B). Because free fluorescein binds to tissues whereas the particle-bound fluorescein does not, use of particle-bound fluorescein provides improved, clear, distinct images of blood vessels without background fluorescence.

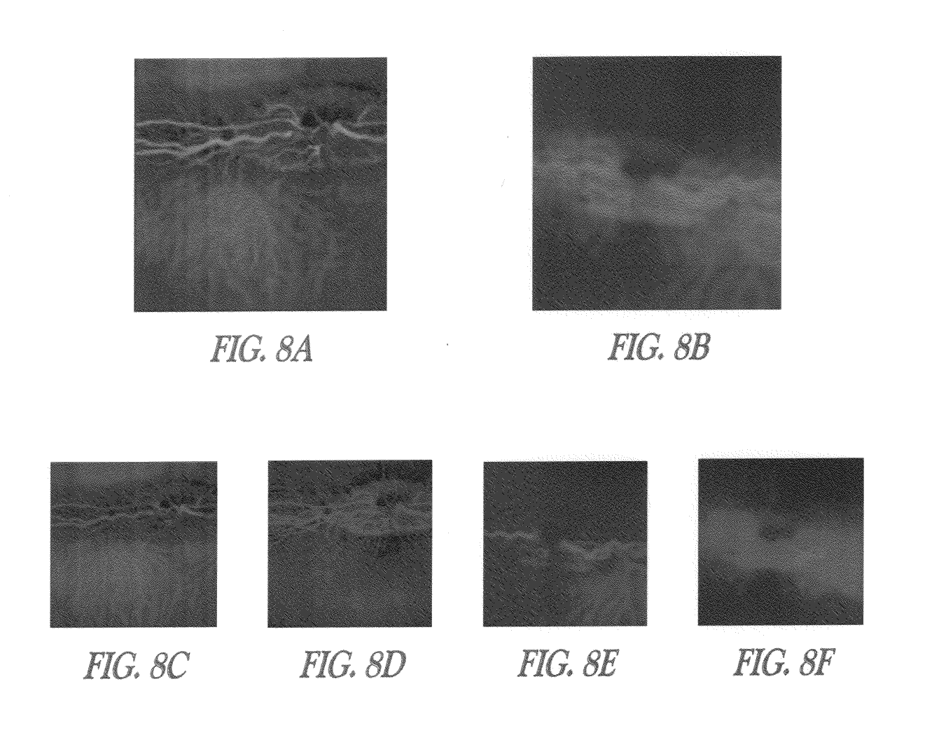

FIG. 8A-F illustrates the selective leakage of different-sized labeled groups in the retina after administration of mixtures of compositions to rabbits. FIG. 8A-C are angiographic images of healthy retinal blood vessels after administration of a mixture of ICG-bound particles and free fluorescein. In FIG. 8B the ICG-bound particles were detected while in FIG. 8C, the fluorescein-bound particles were detected. FIG. 8A shows the fluorescence detected from both ICG-bound particles and free fluorescein in the normal rabbit retina. FIG. 8D-F are angiographic images of mildly dysfunctional retinal blood barriers after administration of a mixture of ICG-bound particles and free fluorescein to a rabbit. Mild retinal blood barrier dysfunction was induced in rabbits by induction of Uvietis through intravitreous injection of LPS (lipopolysaccharide 20 nanograms). In FIG. 8E the ICG-bound particles were detected while in FIG. 8F, the free fluorescein was detected. FIG. 8D shows the fluorescence detected from both ICG-bound particles and free fluorescein in the mildly dysfunctional rabbit retinal blood barrier. Note that in the normal rabbit retina (FIGS. 8A-C) none of the dyes leaked, while in the mildly dysfunctional blood-retinal barrier model, the smaller unbound fluorescein particles leaked from the retinal vessels (the lighter areas outside the vessels in FIGS. 8D and 8F (green in the original)) while the particle-bound ICG (brighter vessels FIGS. 8D & E (red in the original)) did not leak.

Further work was performed with rabbits that illustrated stage 3 dysfunction in the blood-retinal barrier. Such dysfunction was detected with free fluorescein alone or with a combination of indocyanine green-PLGA particles and free fluorescein. Blood-retinal breakdown was induced by inducing uvietis through intravitreous injection of LPS (lipopolysaccharide 20 nanograms). When free fluorescein and indocyanine green-PLGA particles were administered, only free unbound fluorescein leaked through the blood retinal barrier while the indocyanine green particles did not leak.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed:

1. A composition comprising a series of particle groups, each particle group comprising particles with a distinct label that provides a distinct signal and a different mean diameter than other particle groups in the composition.

2. The composition of claim 1, wherein at least one particle group comprises biodegradable particles.

3. The composition of claim 1, wherein at least one particle group comprises non-biodegradable particles.

4. The composition of claim 1, wherein at least one particle group comprises a combination of non-biodegradable and biodegradable materials.

5. The composition of claim 1, wherein each particle group is soluble in an aqueous environment.

6. The composition of claim 1, wherein the labels are covalently attached to particles, adsorbed onto particles, encapsulated within particles, or a combination thereof.

7. The composition of claim 1, wherein the labels are fluorescent, luminescent, infrared, magnetic, radioactive or a combination thereof.

8. The composition of claim 1, wherein at least one particle group comprises poly(lactide), poly(glycolide), poly(lactide-co-glycolide), polyalkylene glycol, poloxamer, polyvinylpyrrolidone, peptide, protein, proteinoid microsphere, lipid, liposome, polysaccharide or a combination thereof.

9. The composition of claim 8, wherein the protein is albumin.

10. The composition of claim 1, wherein the composition comprises particle groups with mean diameters ranging in size from one picometer to three micrometers.

11. The composition of claim 1, wherein the composition comprises particle groups with mean diameters ranging in size from 10 picometers to 900 nanometers.

12. The composition of claim 1, wherein the composition comprises particle groups with mean diameters ranging in size from 1 nanometer to 1 micrometer.

13. The composition of claim 1, wherein the label for at least one particle group comprises fluorescein, fluorescein isothiocyanate, indocyanine green, rhodamine red, pacific blue, texas red, alexa-532, hydroxycoumarin, aminocoumarin, methoxycoumarin, amino methylcoumarin, cascade blue, lucifer yellow, P-phycoerythrin, R-phycoerythrin, lissamine rhodamine B, allophycocyanin, oregon green, tetramethylrhodamine, dansyl, monochlorobimane, calcein, a fluorescent protein or a combination thereof.

14. The composition of claim 1, wherein the label for at least one particle group is a paramagnetic ion.

15. The composition of claim 13, wherein the paramagnetic ion is chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) or erbium (III).

16. The composition of claim 1, wherein the label for at least one particle group is lanthanum (III), gold (III), lead (II), or bismuth (III).

17. The composition of claim 1, wherein the label for at least one particle group is iodine¹³¹, iodine¹²³, iodine¹²⁵, technicium⁹⁹, indium¹¹¹, phosphorus³², rhenium¹⁸⁸, rhenium¹⁸⁶, gallium⁶⁷, sulfur³⁵, copper⁶⁷, yttrium⁹⁰, tritium³ or astatine²¹¹.

18. A kit or article of manufacture, comprising the composition of claim 1, and instructions for using the composition for quantifying blood vessel leakage in a mammal.

19. The kit of claim 18, further comprising a syringe, needle, swab, catheter, or antiseptic solution.

20. A kit or article of manufacture, comprising the composition of claim 1, and instructions for using the composition for monitoring blood flow and/or blood flow rate in a mammal.

21. The kit of claim 19, further comprising a syringe, needle, swab, catheter, or antiseptic solution.

22. A method for quantifying blood vessel leakage in a mammal that comprises: (a) administering the composition of claim I to the mammal; (b) observing whether a signal is exterior to the mammal's blood vessels; and (c) if a signal is emitted exterior to the mammal's blood vessels, determining the type of signal emitted exterior to the mammal's blood vessels to quantify the blood vessel leakage in the mammal.

23. The method of claim 22, wherein the blood vessel leakage comprises leakage through a mammal's blood retinal barrier.

24. The method of claim 22, wherein the blood vessel leakage comprises leakage through a mammal's choroid, retinal pigment epithelium or inner blood retinal barrier.

25. The method of claim 22, wherein the signal is fluorescent, luminescent, infrared, magnetic, radioactive or a combination thereof.

26. The method of claim 22, wherein the label for at least one particle group comprises fluorescein, fluorescein isothiocyanate, indocyanine green, rhodamine red, pacific blue, texas red, alexa-532, hydroxycoumarin, aminocoumarin, methoxycoumarin, amino methylcoumarin, cascade blue, lucifer yellow, P-phycoerythrin, R-phycoerythrin, lissamine rhodamine B, allophycocyanin, oregon green, tetramethylrhodamine, dansyl, monochlorobimane, or calcein.

27. The method of claim 22, wherein determining the type of signal emitted comprises determining the signal's absorption or emission wavelength.

28. The method of claim 22, wherein the blood vessel leakage comprises leakage through a mammal's blood-brain barrier.

29. The method of claim 22, wherein the signal is a paramagnetic or radioactive signal.

30. The method of claim 22, wherein the label for at least one particle group is chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III), erbium (III), lanthanum (III), gold (III), lead (II), bismuth (III), iodine¹³¹, iodine¹²³, iodine¹²⁵, technicium⁹⁹, indium¹¹¹, phosphorus³², rhenium¹⁸⁸, rhenium ¹⁸⁶, gallium⁶⁷, sulfur³⁵, copper⁶⁷, yttrium⁹⁰, tritium³ or astatine²¹¹.

31. The method of any claim 22, wherein the method further comprises quantifying the blood vessel leakage by correlating the signal with the size range of the particle group emitting that signal to identify a pore size in the blood vessel that gives rise to the leakage, and assigning a numerical value to that pore size.

32. A method for quantifying blood-retinal barrier breakdown in a mammal that comprises: (a) intravenously administering a polychromatic fluorescent particle composition to the mammal; (b) observing whether fluorescence is emitted in the mammal's retina; and (c) if fluorescence is emitted, determining the fluorescence absorption or emission wavelength to quantify the blood-retinal barrier breakdown in the mammal; wherein the polychromatic particle composition comprises a series of particle groups, each particle group having a distinct diameter size range and a distinct fluorophore that emits light at a distinct wavelength.

33. A method for observing blood flow and/or blood flow rate in a blood vessel of a mammal comprising administering the composition of claim 1 to a blood vessel of the mammal and detecting at least one signal from at least one particle group of the composition in the blood vessel.

34. The method of claim 33, further comprising correlating the signal with the size range of the particle group emitting that signal to identify diameter of the blood vessel.

35. The method of claim 33, further comprising identifying whether the diameter of the blood vessel changes along the length of the blood vessel or at a later time.

36. The method of claim 33, further comprising identifying whether the blood vessel has a partial blockage.
